(19) United States
(12) Patent Application Publication
(10) Pub. No.: US 2009/0117199 A1
(43) Pub. Date: May 7, 2009

(54) Method of treatment of cancer

(76) Inventors: Timothy C. Scott, Knoxville, TN (US); H. Craig Dees, Knoxville, TN (US); Eric A. Wachter, Oak Ridge, TN (US)

Correspondence Address: COOK ALEX LTD, SUITE 2850, 200 WEST ADAMS STREET, CHICAGO, IL, 60606, US

(21) Appl. No.: 12/315,781

(22) Filed: Dec. 5, 2008

Related U.S. Application Data
(63) Continuation-in-part of application No. 11/951,800, filed on Dec. 6, 2007.
Application No. 11/951,800 is a continuation-in-part of application No. 09/900,355, filed on Jul. 6, 2001.
Application No. 09/900,355 is a continuation-in-part of application No. 09/130,041, filed on Aug. 6, 1998, now abandoned.
Application No. 09/130,041 is a continuation-in-part of application No. 09/635,276, filed on Aug. 9, 2000, now abandoned.
Application No. 09/635,276 is a continuation-in-part of application No. 09/216,787, filed on Dec. 21, 1998, now Pat. No. 6,331,286.
Application No. 09/900,355 is a continuation-in-part of application No. 09/799,785, filed on Mar. 6, 2001, now Pat. No. 7,390,668.
(60) Provisional application No. 60/218,464, filed on Jul. 14, 2000.

(51) Int. Cl. A61K 33/42; A61K 33/14; A61P 35/00

(52) U.S. Cl. 424/601; 424/680

(57) ABSTRACT

A method for the treatment of cancer, particularly a metastatic melanoma or a neoplastic lesion, the method comprising intralesional administration of a hydrophilic vehicle comprising 4,5,6,7-Tetrachloro-2′,4′,5′,7′-tetraiodofluorescein, or a physiologically acceptable salt thereof, at a concentration of about 0.1 w/v % up to about 20 w/v % and an electrolyte comprising at least one cation selected from the group consisting of sodium, potassium, calcium and magnesium and at least one anion selected from the group consisting of chlorine, a phosphate and a nitrate, wherein the electrolyte is at a concentration of between about 0.1 w/v % and about 2 w/v % and the pH of the solution between about 4 to about 10.

This application is a continuation-in-part of U.S. application Ser. No. 11/951,800 filed Dec. 6, 2007 which is a continuation-in-part of U.S. application Ser. No. 09/900,355 filed Jul. 6, 2001 which claims the benefit under 35 USC §119(e) of U.S. application 60/218,464 filed Jul. 14, 2000. The '355 application is also a continuation-in-part of U.S. application Ser. No. 09/130,041, filed on Aug. 6, 1998; U.S. application Ser. No. 09/635,276 filed on Aug. 9, 2000 which is a continuation-in-part of U.S. application Ser. No. 09/216,787 filed Dec. 21, 1998; and U.S. application Ser. No. 09/799,785 filed on Mar. 6, 2001, which are herein incorporated by reference in their entirety.

The present invention relates to a method for the treatment of cancer. In particular, the present invention relates to a method of treatment of Stage III and IV metastatic melanoma.

### BACKGROUND OF THE INVENTION

The present invention will be described with particular relevance to melanoma. However, the method of the present invention may also find application for the treatment of other hyperproliferative diseases including, breast cancer, liver cancer, prostate cancer and small cell lung cancer, and no limitation is intended thereby.

Malignant melanoma is the most serious form of skin cancer and accounts for 80% of skin cancer deaths. Australia has the world's highest incidence rate and represents a lifetime risk for one in 25 Australian men.

The extent of spread of a disease is described by stages. Stage 0 melanoma is a very early stage disease known as melanoma in situ. Patients with melanoma in situ are classified as Tis (tumor in situ). The tumor is limited to the epidermis with no invasion of surrounding tissues, lymph nodes, or distant sites. Melanoma in situ is considered to be very low risk for disease recurrence or spread to lymph nodes or distant sites. Treatment is by surgical excision with a margin of healthy skin.

In stage I melanoma, the tumor has penetrated in to the skin by less than 1 mm but has not spread. Treatment is by wide local excision and the probability of disease free survival in five years is between 90 to 95%.

Stage II melanoma describes a tumor that has penetrated more than 1 mm into the skin but has not spread. Wide local excision is the preferred treatment. However, excision at this stage carries a much higher risk and less favourable prognosis than excision of a Stage I tumor.

Stage III melanoma is characterized by the existence of one or more nodal, intransit or satellite metastasis but has not spread to distant sites. Intransit metastases are distant from the primary tumor but not reaching the draining nodal basin. Satellite metastases are intralymphatic extensions of the primary tumor and are typically found closer to the primary tumor than intransit metastasis. Five year survival for stage III patients ranges from approximately 24% (gross nodal disease) to 80% (microscopic nodal disease).

Stage IV melanoma is when the disease has spread to distant sites. Survival of stage IV melanoma drops to approximately 10%,

Standard treatment for easily removable Stage III tumors is wide area excision together with removal of lymph nodes. Adjunct treatment such as radiotherapy and chemotherapy and for regional limb metastases, regional infusion of melphalin or other chemotherapeutic agents may also be given. However, in some cases, surgery is contraindicated due to the number and/or location of tumors and other treatment options must be considered. Unfortunately, response levels for these other options are not high. For example, melanoma is largely resistant to radiation therapy. Systemic chemotherapy also has modest response rates. The most effective chemotherapy regimen to-date is the single-agent dacarbazine (DTIC), which is only successful in 10-15% of cases. Two combination chemotherapy regimens commonly used in the treatment of patients with advanced-stage melanoma are the cisplatin, vinblastine, and DTIC (CVD) regimen and the Dartmouth regimen, which is a combination of cisplatin, DTIC, carmustine, and tamoxifen.

When melanoma occurs in the extremities, chemotherapy agents may be delivered via hyperthermic isolated limb perfusion. With this technique, blood vessels are accessed surgically, the blood flow to and from the limb is stopped using a tourniquet, and a warmed solution of chemotherapy drug is administered directly into the blood of the limb, allowing higher doses of drugs to be dispensed than with systemic treatment. A less invasive regional therapy is isolated limb infusion whereby vascular access is gained via a percutaneous route in the groin.

Another treatment option is intralesional therapy (IL) in which a chemotherapeutic agent is injected directly into the tumor. Bacille Calmette Guerin (BCG) was one of the earliest reagents used for IL therapy. A review of data from 15 trials found that 19% complete response (CR) and 26% partial response (PR) with extended survival in 13% of stage III patients.

IL interferons (IFN) have yielded mixed results ranging from a report of 45% objective response rate (ORR, 31% CR+14% PR) for IFN∝ to either no result of transient response with IFNγ. Both regimes produced significant toxicity and side effects.

IL interleukin-2 appears to be the most promising IL therapy to date with an ORR in 83% of patients (62% CR+21% PR) receiving 2-3 weekly IL treatments. Some patients reported flu like symptoms and some authors noted that although new lesions appeared during the course of treatment, some patients experienced a marked slowing of the appearance of new cutaneous lesions.

IL therapy with cisplatin or IL cisplatin with electroporation has yielded results ranging from 38% ORR (19% CR+19% PR) to 53% ORR (47% CR+7% PR). However, the ORR reported for lesions with a median diameter of 0.6 cm of 53% decreased to 44% for lesions having a median diameter of 3.0 cm.

Substantial efficacy has been reported upon a single electrochemotherapy treatment with IL bleomycin. However, as with cisplatin, response was generally reduced in larger tumors. It may be appreciated that there remains a need for alternative methods for the treatment of hyperproliferative diseases and in particular stage III and IV melanoma.

### SUMMARY OF THE INVENTION

According to a first embodiment of the present invention, there is provided a method for the treatment of a metastatic melanoma, the method comprising intralesional administration of a hydrophilic vehicle comprising 4,5,6,7-Tetrachloro-2′,4′,5′,7′-tetraiodofluorescein (i.e. Rose Bengal), or a physiologically acceptable salt thereof, at a concentration of about 0.1 w/v % up to about 20 w/v % and an electrolyte comprising at least one cation selected from the group consisting of sodium, potassium, calcium and magnesium and at least one anion selected from the group consisting of chlorine, a phosphate and a nitrate, wherein the electrolyte is at a concentration of between about 0.1 w/v % and about 2 w/v % and the pH of the solution between about 4 to about 10.

According to a further embodiment of the present invention, there is provided a method for the treatment of a neoplastic lesion, the method comprising intralesional administration of a hydrophilic vehicle comprising 4,5,6,7-Tetrachloro-2′,4′,5′,7′-tetraiodofluorescein, or a physiologically acceptable salt thereof, at a concentration of about 0.1 w/v % up to about 20 w/v % and an electrolyte comprising at least one cation selected from the group consisting of sodium, potassium, calcium and magnesium and at least one anion selected from the group consisting of chlorine, a phosphate and a nitrate, wherein the electrolyte is at a concentration of between about 0.1 w/v % and about 2 w/v % and the pH of the solution between about 4 to about 10.

The neoplastic lesion may be any lesion associated with a hyperproliferative disease. The present inventors have surprisingly shown that the a hydrophilic vehicle comprising 4,5,6,7-Tetrachloro-2′,4′,5′,7′-tetraiodofluorescein, or a physiologically acceptable salt thereof, at a concentration of about 0.1 w/v % up to about 20 w/v % and an electrolyte comprising at least one cation selected from the group consisting of sodium, potassium, calcium and magnesium and at least one anion selected from the group consisting of chlorine, a phosphate and a nitrate, wherein the electrolyte is at a concentration of between about 0.1 w/v % and about 2 w/v % and the pH of the solution is up to about 7, has activity against a number of different cancers in the mouse model. This is indeed surprising as it is well known that cancer treatment is generally specific to the type of cancer cell.

The term ‘physiologically acceptable salt’ refers to any non-toxic alkali metal, alkaline earth metal, and ammonium salts commonly used in the pharmaceutical industry, including the sodium, potassium, lithium, calcium, magnesium, barium ammonium and protamine zinc salts, which can be prepared by methods known in the art. Preferably, the salts are sodium, potassium, calcium and ammonium in either the mono of dibasic salt form.

Especially preferred is the disodium salt of Rose Bengal. It is known that Rose Bengal is almost exclusively removed from the blood by the liver and is rapidly excreted from the bile in unmetabolized form. This property has led to the use of ¹³¹I Rose Bengal as an intravenous diagnostic agent for liver function. Typical intravenous diagnostic doses are between 0.09 mg to 0.4 mg.

Rose Bengal disodium has the following formula:

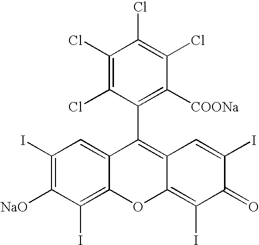

Previous work by the present inventors (WO 02/05812) reports that Rose Bengal exhibits preferential uptake into cancer cells but is essentially excluded from normal cells.

Subsequent to this work, the present inventors have now surprisingly and unexpectedly discovered that the nature of the vehicle in which 4,5,6,7-Tetrachloro-2′,4′,5′,7′-tetraiodofluorescein, or a physiologically acceptable salt thereof, is administered can significantly influence the degree of partitioning into tumor cells. In particular, the present inventors have surprisingly discovered that at an electrolyte concentration of between about 0.1% and about 2.0%, partitioning into tumor cells may rapidly be increased. This approach differs from that suggested in WO 02/05823 which teaches optimizing the facility with which halogenated xanthenes target specific tissues by attachment of functional derivatives so as to change the chemical partitioning and/or biological activity of the agent.

An approximation as to an agent's potential for tissue accumulation can be estimated based upon the partition coefficient Kₚ. This in vitro parameter is purported to have predictive values relating to in vitro delivery at the cellular level. In particular, a value greater than unity is considered to indicate agents capable of localizing in tissue, and thereby being capable of exhibiting enhanced chemotherapeutic efficacy in such tissue. The present inventors surmise that values much greater than approximately 50-100 may indicate excess lipophilicity (tendency to accumulate in organic environments) that may compromise delivery of an agent in a desirable aqueous (i.e., hydrophilic) formulation. Kₚ is determined by measuring the ratio of equilibrium concentrations of an agent in a lipophophilic phase (n-octanol) contacted with an aqueous phase. The present inventors have found that it is preferred that the pH of the solution is in the range of between about 4 to about 10, and more preferably between about 5 to about 9, to yield maximum solubility of the agent in an aqueous vehicle and assure compatibility with biological tissue. A particularly preferred pH is between about 4 to about 7, preferably between about 5 to about 7, more preferably between about 6 to about 7, and still more preferably between about 6 to about 6.8. At these pH values, 4,5,6,7-Tetrachloro-2′,4′,5′,7′-tetraiodofluorescein generally remains in dibasic form, rather than the water insoluble lactone that forms at low pH.

The pH of the solution may be regulated or adjusted by any suitable means known to those of skill in the art. The solution may be buffered or the pH adjusted by addition of acid or base or the like. As 4,5,6,7-Tetrachloro-2′,4′,5′,7′-tetraiodofluorescein, or a physiologically acceptable salt thereof, is itself a weak acid, depending upon its concentration and/or electrolyte concentration, the pH of the solution may not require the use of a buffer and/or pH modifying agent. It is especially preferred, however, that the vehicle does not contain any buffer, allowing the medicament to conform to the biological environment once administered.

A still further surprising and unexpected result is that Kₚ is also dependent upon electrolyte concentration with the Kₚ value increasing with electrolyte concentration. Comparative values of Kₚ and electrolyte concentration are provided in Table 1 of Example 1. Particularly preferred concentrations of electrolyte are between 0.5-1.5%, and even more preferably at a concentration of about 0.8-1.2% and most preferably at a concentration of about 0.9%, this latter concentration being especially preferred in that it corresponds to an approximately isotonic solution.

In a further embodiment of the present invention, the electrolyte is sodium chloride.

Electrolytes at such levels increase the osmolality of the vehicle. Thus, as an alternative to specifying a range of electrolyte concentrations, osmolality may be used to characterize, in part, the electrolyte level of the medicament. It is preferred that the osmolality of the medicament be greater than about 100 mOsm/kg, and more preferably that the osmolality of the medicament be greater than about 250 mOsm/kg and most preferably that it is about 300-500 mOsm/kg.

The present inventors have also surprisingly found that direct injection into diseased tissue (i.e., intralesional injection) is a particularly effective means of administration of 4,5,6,7-Tetrachloro-2′,4′,5′,7′-tetraiodofluorescein for treatment of focal disease since it concentrates and maximizes the therapeutic effects of the medicaments in target tissue whilst minimizing potential for deleterious effect elsewhere in the patient.

Accordingly, one embodiment of the method of the present invention involves intralesional administration of the active agent. The concentration of agent and/or dose will be dependent upon factors including, but not limited to, tumor, size, number and location, age of patient and any other relevant medical factor. For visceral or other internal lesions, such as liver cancer, the intralesional administration may be by percutaneous or intraoperative administration.

The present inventors have also found that 4,5,6,7-Tetrachloro-2′,4′,5′,7′-tetraiodofluorescein or salt thereof concentrations above about 1% to 3% are particularly useful for chemotherapeutic use, since lower concentrations are generally insufficient to elicit necrosis or other desired mechanisms of death in target tissues. Thus, in a preferred embodiment, the concentration of 4,5,6,7-Tetrachloro-2′,4′,5′,7′-tetraiodofluorescein or salt thereof is in the range of from about 3% to about 20%. In another embodiment, the concentration of 4,5,6,7-Tetrachloro-2′,4′,5′,7′-tetraiodofluorescein or salt thereof is from about 3% to about 10%. In another embodiment, the concentration of 4,5,6,7-Tetrachloro-2′,4′,5′,7′-tetraiodofluorescein or salt thereof is from about 10% to about 20%. In still another embodiment, the concentration of 4,5,6,7-Tetrachloro-2′,4′,5′,7′-tetraiodofluorescein or salt thereof is about 10%. The present inventors have surprisingly found that at these concentrations, not only can an efficient therapeutic response be obtained, but the solution is also highly stable and can be readily handled both in manufacture and use. These preferred concentrations may be weight to volume (w/v) or weight to weight (w/w)

Typical dosages of the hydrophilic vehicle administered by intralesional administration range from between 0.1 mL/cc lesion volume to about 2 mL/cc lesion volume, most preferably between about 0.25 mL/cc to about 0.75 mL/cc lesion volume. Such doses typically correspond to a patient dose of between about 10 mg to about 1500 mg, (which are significantly higher than those doses used for diagnostic liver tests).

The inventors have further found that such intralesional injection is optimally effected using a fine gauge needle for injection, preferably 22-24 gauge or smaller, and more preferably 26 gauge or smaller, to minimize leakage of injected medicament via the needle track. It is further preferred that such injection be performed using a minimum of punctures into the injected tissue, whereby the needle is inserted into the injected tissue a minimum number of times and then, using a “fanning” or similar technique, starting at the margin and slowly withdrawing the needle during each fractionated injection. Multiple injection tracks may thereby be applied, using a single puncture when possible and while re-injecting at multiple angles into the treated lesion while minimizing tearing and leakage until the entire tissue volume is uniformly infiltrated. Alternatively, an injection device having several tips adapted for simultaneous injection of infusion to multiple locations within the target tissue, such as that described by Edwards et al (U.S. Pat. No. 7,150,744) may be used.

Since the solution is for IL delivery, it is further preferred that that the solution be sterile, such as required for conformance to U.S. Pharmacopeia (USP) test <71>, and further that it contains negligible levels of pyrogenic material, such that it conforms to USP <85> (limulus amebocyte lysate assay) or to USP <151> (rabbit pyrogen test), or to substantially equivalent requirements, at a pyrogen level to not more that (NMT) 10 endotoxin units (EU) per mL. Moreover, the solution should conform to requirements limiting content of particulate matter as defined in USP <788> (i.e., NMT 3000 particulates greater than 10 microns in size, and NMT 300 particulates greater than 25 microns in size, per container) or substantially equivalent requirements. Each of these documents is incorporated herein by reference.

Still further, the inventors have found that a hydrophilic vehicle is preferred for the 4,5,6,7-Tetrachloro-2′,4′,5′,7′-tetraiodofluorescein or salt thereof to maximize preference for partitioning into tissue. Accordingly, it is preferred that the vehicle contain a minimum of non-hydrophilic components that might interfere with such partitioning. It is preferred that the hydrophilic vehicle is water, and it is most preferred that this vehicle consists substantially of water.

The inventors have found that such medicaments as described herein are optimally packaged in glass vials having a capacity of approximately 1 to 10 mL, and more preferably approximately 5 mL. Such capacities are well suited as unidose forms (i.e., single use packages) for IL treatments.

In a preferred embodiment, the formulation is not buffered. In this case, it is preferred that such containers be made of the USP Type I (low extractable or chemically resistant borosiciliate) or USP Type II (low-extractable soda lime) glass and that the inside surface of such glass containers be surface treated to reduce surface alkalinity of the container that could adversely affect pH or long-term stability. Typical surface treatment applicable to such containers is described in USP<661>. The inside of such surface-treated glass containers should be rinsed with a suitable solvent, such as distilled water one or more times prior to filling in order to remove any residue of such surface treatment. The containers should also be depyrogenated prior to filling, for example, by heating to 250° C. or higher for several hours or more, and should be sterile or sterilized prior to filling using methods common in the field. If is further preferred that such containers have a minimum neck size, for example, of less than 2 mm and more preferably 10 mm or less, to reduce surface area of the closures of the containers (and hence exposure of the medicament to the closures).

The inventors have further found that a septum-type closure, composed preferably of a pharmaceutical grade elastomeric material with a Teflon or similar inner coating, is particularly suitable for use since it facilitates insertion of a needle into the container for withdrawal of a dose of medicament while exhibiting minimal potential for interaction with the container contents.

It is also preferred that the vehicle does not include any preservatives. The inventors have found that it is generally preferable to avoid use of preservatives, many of which may deleteriously interfere with the medicament or formulation thereof, or my complex or otherwise interact with or interfere with the delivery of the Rose Bengal. To the extent that a preservative may be used, the inventors have found that imidurea is preferred as it does not interact with Rose Bengal, either in the medicament, or upon administration, nor to deleteriously affect the medicament formulation.

The method of the present invention may find particular application for the treatment of patients with stage III or IV metastatic melanoma and in particular the treatment of satellite and/or Intransit lesions.

### BRIEF DESCRIPTION OF THE FIGURES

FIG. 1 is a graph showing the variation of Kp with pH;

FIG. 2 is a graph of showing the solubility of Rose Bengal (RB) in different saline concentrations and

FIG. 3 shows the solubility of RB in 0.9% saline.

FIG. 4 is a graph of Rose Bengal (RB) in tumor tissue, peritumor tissue and blood following a single IL injection of 1% RB in A375 tumor versus time post injection; and

FIG. 5 is a graph of RB in tumor tissue, peritumor tissue and blood following a single IL injection of 6% RB in A375 tumor versus time post injection.

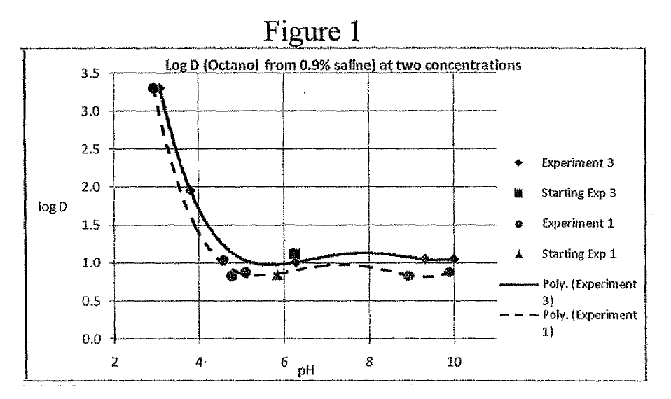

### DETAILED DESCRIPTION OF THE INVENTION

### 1. Partitioning Coefficient Studies—Effect of Electrolyte Concentration

The partitioning coefficients of Rose Bengal (RB) were determined by partitioning a solution of 0.5 mg/mL Rose Bengal in 0%, 0.5%, 1.5% and 2.5% saline with 1-octanol. After mixing, the agent was allowed to partition for approximately 1 day. Based on absorbance measurements at 550 nm for the aqueous phase and 564 nm for the organic phase, the percentage of agent in each phase was obtained. The results are shown in Table 1 below:

TABLE 1

| | | Partitioning coefficient |
|---|---|---|
| Saline solution % | % Partitioning in Octanol | (Kp) |
| 0.00% | 75.79 ± 1.75 | 3.14 ± 0.29 |
| 0.50% | 95.13 ± 1.43 | 20.84 ± 6.77 |
| 0.90% | 98.52 ± 0.26 | 68.17 ± 4.43 |
| 1.50% | 97.77 ± 0.25 | 44.25 ± 5.52 |
| 2.50% | 99.35 ± 0.28 | 215.12 ± 105.22 |

It may be seen that with increasing saline content partitioning into octanol generally increased gradually, and increased dramatically above 1.5%.

### 2. Partitioning Coefficient Studies—Effect of pH

The partition coefficient of RB was assessed over a range of pH values. The experiments were conducted by titrating RB solutions, measuring pH, then placing aliquots of the solutions into vials containing both organic (1-octanol) and aqueous (0.9% saline) phases. After mixing, the agents were allowed to partition for approximately 1 day. Based on absorption measurements (at the same wavelengths used in Experiment 1), the percentage of agent in each phase, the Kp and LogD (log of Kp) was reported.

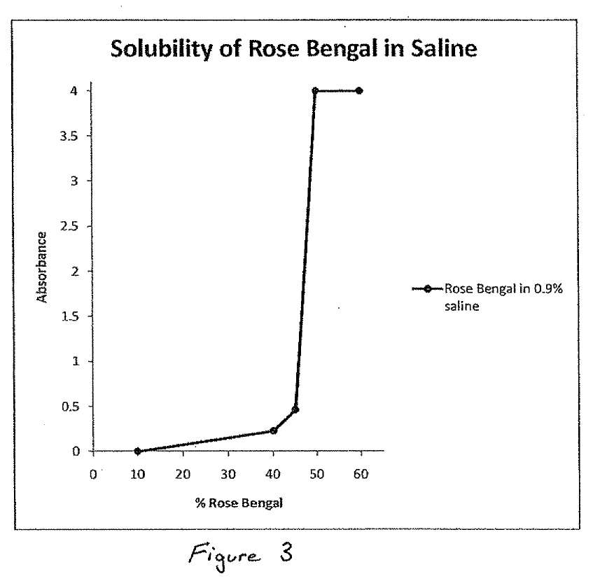

The results are shown in FIG. 3. Experiment 1 was conducted using a stock solution of 0.42 mg/mL RB, whereas Experiment 3 was conducted using a concentration of 0.08 mg/mL. The solubility of RB in octanol was estimated to be 0.195 mg/mL. The results were therefore slightly suppressed in Experiment 1 where the concentration exceeded the solubility of RB in 1-octanol.

The results show that the partitioning coefficient is consistent over the preferred pH range of between about 4 to about 10. Below a pH of about 4.4, the partitioning coefficient rapidly increases. However, this is accompanied by almost complete insolubility of RB in the aqueous phase. Below a pH of 4.4, RB spontaneously converts to the lactone form which has poor solubility in water. Whilst not wishing to be bound by theory, it is postulated that this conversion to the insoluble lactone may result in the compound becoming trapped in intracellular compartments having low pH. For example, lysozoymes have a pH of about 4.8.

3. Critical solubility of Rose Bengal (RB) in Saline

The objective of this study was to determine the limit of solubility (critical solubility) of Rose Bengal in sodium chloride solution.

Solutions of RB at different concentrations were prepared at different sodium chloride concentrations. Saline 0.9% USP sodium chloride solution from Hospira, having a pH of 5.5, was used as a stock solution. Solutions having different sodium chloride concentrations were obtained by the addition of calculated amounts of sodium chloride.

The prepared RB/sodium chloride solutions were shaken for 1 hour and absorbance measurements were taken at 1000 nm to determine cloudiness. 1000 nm was chosen as this is in the near infra red where RB has negligible absorbance. Solution cloudiness was difficult to detect visually due to the very dark red color.

The following solutions were evaluated:
1. 20% RB in saline concentrations of 1.0%, 1.5%, 1.6%, 1.7%, 1.8%, 1.9%, 2.0%, 2.5%, 2.6%, 2.7% and 2.9%;
2. 15% RB in saline concentrations of 0%, 2.5%, 3.0% and 3;
3. 10% RB in saline concentrations of 2.0%, 2.5%, 3.0% and 3.2%; and
4. 40%, 45%, 50%, 55% and 60% solutions of RB in 0.9% saline.

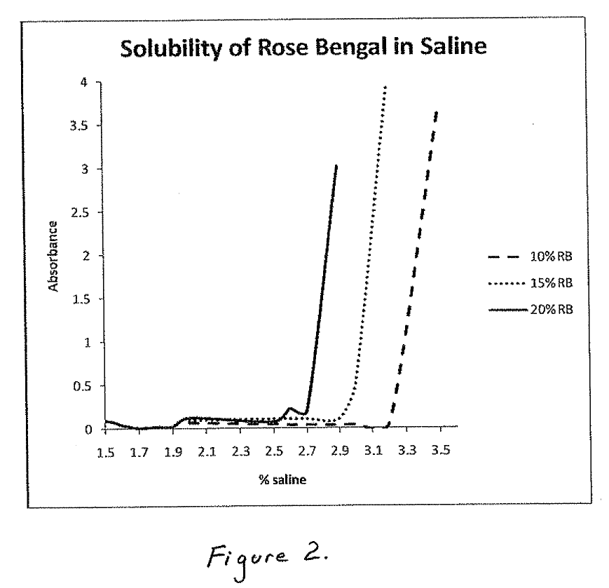

The results are shown in FIGS. 2 and 3. FIG. 2 shows that the critical solubility for 20% RB is between 2.7% and 2.9% aqueous concentration of saline. The approximate critical solubility of 15% RB is between 2.8% and 3% and that for 10% RB is between 3% and 3.3%. FIG. 3 shows that a concentration of up to about 45% RB can be achieved in 0.9% saline.

These results show that RB solutions are stable within the therapeutic amounts as used in the methods of the present invention. Clearly solution stability is important for medicinal use.

4 In vitro Studies

The effects of a single IL injection of RB into A375 human melanoma xenograft tumors growing in nude mice were evaluated.

4.1 Animals

Chemoablation studies were conducted using female nu/nu mice (Harlan), 6-9 weeks of age and approximately 20 g at study Day 1.

The local tolerance study was conducted using female Crl:SKH-1-hrBR hairless mice (Charles river), at least 6 weeks of age and 19-27 g at study Day 1.

4.2 Tumor Implantation

Each mouse in Chemoablation study groups received a single subcutaneous implantation of the A375 melanoma tumor line. This consisted of implantation of either (1) a 1 mm³ fragment of tissue in the flank or (2) subcutaneous injection of 100 μL of an A375 cell suspension into the flank. Treatment was initiated when the implanted tumor reached a size of approximately 70 mg (where tumor weight, in mg, was estimated using the formula (w²×l)/2 in which w is width, and l is length in mm of the tumor).

4.3 Materials

RB (Aldrich product No. W1843) in isotonic saline; concentrations of 0.1% RB, 1%, RB, 1%, RB, 6% RB and 10% RB (w/v) was studied. Saline was used for the injection control group.

4.4 Experimental Procedure

Chemoablation

Treatment Plan: Mice were divided into treatment groups on study Day 1. Group assignments are summarized in Table 1. RB treatment groups received a single IL injection of 0.1 mL RB in saline; the injection control group received a single IL injection of 0.1 mL saline. Growth control and sham treatment groups received no injection. All dosing was performed on Study Day 1. Follow up examination of the mice was conducted at regular intervals, and the test terminated on either Study day 65 or Study Day 85.

Each animal was euthanized when its A375 neoplasm reached a size of 2.0 g. Animals whose tumors did not reach 2.0 g were euthanized at study termination.

Treatment was assessed for occurrence of complete tumor regression (CR), partial tumor regression (PR) or stable disease (SD). The duration of CR, PR, or SD response was recorded throughout each study.

The Log-Rank test was used to determine the statistical significance of any difference in survival experienced between treatment and control groups. The Fishers Exact Test was used to determine the statistical significance of differences in numbers of mice exhibiting positive tumor response (total number exhibiting CR, PR, or SD) between treatment and control groups. SigmaStat 3.0 (SPSS Inc) was used for all statistical analysis. Graphs were prepared using sigmaStat 3.0 or sigma Plot 2000 (SPSS Inc.) ver 6.00.

Group assignments are shown in Table 2

TABLE 2

| | | | |
|---|---|---|---|
| | | | Dosage Level |
| Group | N | Agent | (mg/kg) |

| A | 18 | Growth control | | 0 |
|---|---|---|---|---|
| B | 6 | Saline | | 0 |
| C | 5 | 0.1% | RB | 5 |
| D | 12(a) | 1% | RB | 50 |
| E | 17 | 3% | RB | 150 |
| F | 6(a) | 6% | RB | 300 |
| G | 5 | 10% | RB | 500 |

(a)sixteen additional mice were included in these groups for collection of samples for pharmacokinetic and toxicokinetic analysis

### A. Local Tolerance

RB treatment groups received a single subcutaneous (SC) injection of Rose Bengal in saline; the control group received a single SC injection of saline. All dosing was performed on Study Day 1. Follow-up examination of mice was conducted at regular intervals and the test terminated on either day 4 (3 animal from each group) or Day 15 (the remaining three animals). Group assignments are shown in Table 3:

TABLE 3

| | | | | Dosage | Dosage |
|---|---|---|---|---|---|
| | | | | Volume | Level |
| Group | N | Agent | | (mL) | (mg/kg) |
| 1 | 6 | Control | | 0.4 | 0 |
| | | (Saline) | | | |
| 2 | 6 | 1% | RB | 0.1 | 50 |
| 3 | 6 | 5% | RB | 0.1 | 250 |
| 4 | 6 | 10% | RB | 0.1 | 500 |
| 5 | 6 | 10% | RB | 0.25 | 1250 |
| 6 | 6 | 10% | RB | 0.4 | 2000 |

Clinical observations were recorded daily (including dermal scoring using the Draize scoring system). Upon sacrifice, animals were subjected to a comprehensive necroscopy. Tissue samples from the injection site (and surrounding tissue) were obtained and examined histologically.

4.5 Results

### Control Groups:

Control groups exhibited progressive tumor growth and reached the 2.0 g study endpoint with calculated MST values of 26.7±2.7 days (Growth Control) and 27.62±2.7 days (Saline Injection control). A single neoplasm in Group A exhibited stable disease (SD), while all other control animals the implanted neoplasm exhibited steady, rapid progression to endpoint size. Neither survival nor outcome of the Injection control Group (Group B) was statistically different from the Growth control Group (Group A).

RB Treatment Groups

Animals receiving 0.1% RB (Group C) exhibited tumor growth response that was qualitatively and statistically equivalent to the control groups. In contrast, survival analysis (using the Log-Rank Test) indicated a progressive increase in MST for RB concentrations of 1% and higher, with a statistically significant increase (relative to growth control) for both 3% and 10% RB, The small cohort of animals treated with 6% RB also exhibit increased survival but are not statistically different-than growth controls, presumably due to the small size of the groups (N=6).

Efficacy data is summarized in Table 4.

### Clinical Response

A large fraction of tumors in the RB treatment groups in both studies (i.e., 1% RB and higher) exhibited localized tumor necrosis with rapid eschar formation at the injection site. This was followed by complete healing of the eschar with no prolonged exudation of fluid or other ling term adverse events.

### Toxicology

All tumour treatments were well-tolerated with respect to systemic toxicity. The noted anti-neoplasmic effect was highly localized and was not associated with evidence of systemic toxicity.

### Biodistribution

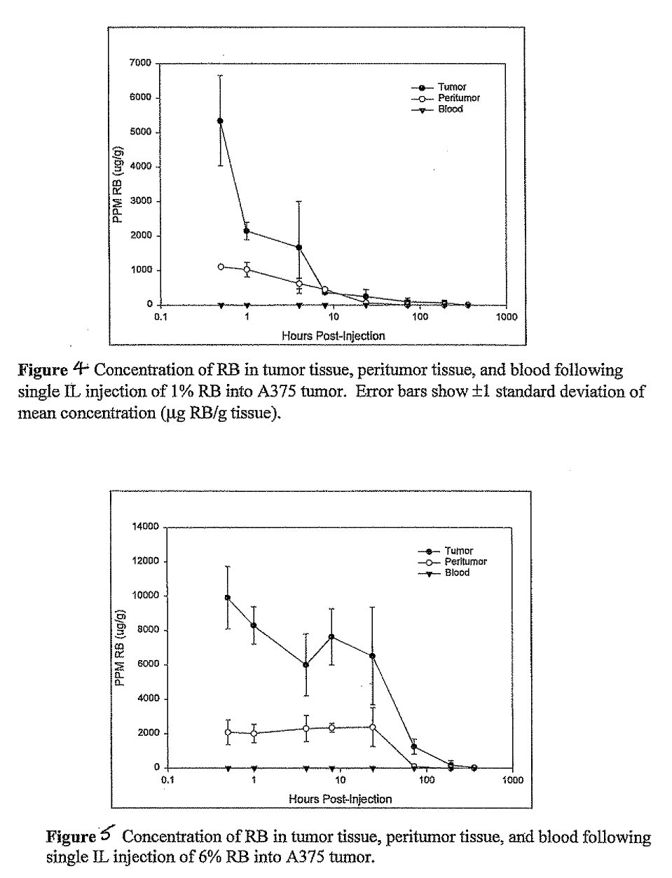

Pharmacokinetic and toxicokinetic data from RB-treated tumors illustrates that a single IL injection of RB leads to localized retention of drug tumor tissue. This is illustrated in FIGS. 4 and 5, which show concentration of RB in tumor tissue, peritumor tissue and blood for time points ranging from 0.5 hr to 360 hr (15 days) post injection.

Peak intralesional levels are noted at the initial time-points in both figures. Markedly elevated levels persist in tumor samples obtained at all time points within at least 8 days post-injection, Level of RB in peritumor tissue exhibit similar trends, although at generally much lower levels. Mean levels in blood never exceed 3 μg/g, indicate that low levels of RB released from the IL depot are rapidly excreted (note that the half-life of RB in the murine blood stream is approximately 30 min). Although there is no direct correlation between tumor and peritumor levels of RB and injected RB concentration, the levels of RB in blood during at least the first 8 hours post-injection are proportional to IL dose.

### Histology

Terminal Sacrifice: Histological examination of tumor and peritumor tissue from mice obtained at terminal sacrifice was characterized by minimal perilesional inflammation and damage, with no dose-related trends noted in any such effects. Moreover, no residual RB was noted in perilesional tissue.

Inflammation and damage of tumor tissue exhibited a possible dose response, as illustrated by the data in Table 6. The composite response comprises an average of damage+necrosis/2. This composite value as well as the respective value for mean severity of necrosis, is elevated for the 1%, 3% and 6% RB treatment groups, supporting the existence of a positive dose response. Values for the 10% RB treatment group are similar to those of the control groups, and may result from a combination of statistical variability and small group size.

### Local Tolerance

Clinical Outcome: Mice treated with up to 500 mg RB/kg body weight (i.e., corresponding to a 10% treatment dose) exhibited mild side effects primarily limited to localized discoloration of the injection site on Study Day 15. This discoloration appears to have resulted from residual RB remaining at the injection site and in surrounding tissue. Swelling was also noted in the injection site for most animals treated with 250 and 500 m mg/kg.

In contrast to these mild effects, mice receiving dosages of 1250-2000 mg/kg exhibited agent related massive necrosis at the injection site; there were also two fatalities in the 2000 mg/kg study group (on days 2 and 3).

Histology: Animals receiving <500 mg RB kg body weight exhibited focal necrosis at the injection site on days 4 and 15, markedly reduced incidence and severity at the later time point. Inflammation was restricted to the perimeter of the necrotic regions. On Study day 15, only Groups 5 and 6 exhibited massive necrosis at the injection site.

Other Lesions: the two fatalities in Groups 6 (i.e., 2000 mg/kg) exhibited mild to moderate degeneration of the liver, and one of the two had mild degeneration of the kidney. Both effects were considered to be agent related. No systemic effects were noted for animals in the lower dosage groups.

Conclusions

Intralesional injection of RB at concentrations up to 10% (i.e., 500 mg/kg) was well tolerated by all animals. Injection of tumors using concentrations of 1% RB and higher resulted in increasing survival and enhanced outcome relative to growth control and saline injection control groups, with the greatest effects noted at 10% RB. Pharmacokinetic and histological data support these clinical observations, indicating that IL injection of RB leads to prolonged, localized retention of RB at high concentration, with minimal release to adjacent peritumor tissue or into the blood stream. This local depot appears to afford selective Chemoablation of tumor tissue that, if successful, leads to localized eschar at the treatment site with no prolonged localized or systemic adverse affects.

In contrast to the marked anti-neoplastic effects observed in these studies, subcutaneous injection of RB into normal tissue elicited only mild clinical effects at dosages of up to 500 mg/kg (i.e., 10%), with no histological evidence of necrosis at dosages up to 250 mg/kg at 1 days post administration. These findings support the observed safety profile of RB upon IL injection, indicating that IL injected RB poses limited safety risks to normal tissue at the injection site or elsewhere in the subject.

It will be appreciated that in the local tolerance studies RB was subcutaneously injected into normal tissue, such that normal tissue was exposed to the full dose of RB. This is to be compared with IL injection in which RB is localized in the tumor and significantly less RB congregates into the normal peritumor tissue.

4.6 Other Cell Lines

The results of further studies are summarized in Table 6.

TABLE 6
Tumor Response of Tumor Cell Lines to IL 10% RB in Saline at Neutral pH

| | | | | |
|---|---|---|---|---|
| Report | | Tumor | | |
| Number | Species | Cell Line | Dosage | RESULTS |

Breast Carcinoma Cell Lines

| | | | | |
|---|---|---|---|---|
| 020110.01B | BALB/c | Human: | 10% RB IL | IL RB completely ablated MCF-7 |
| | Nude Mice | MCF-7 | 25-50 μL/tumor | [ER+] (4/4), MCF-7 [ER−] (4/4), |
| | 4/group | [ER+] | Qd × 1 | and HTB-133 (4/4) tumors within |
| | | MCF-7 | | 21 days. Most long Passage T- |
| | | [ER−] | | 47D [ER−] tumors were ablated |
| | | HTB-133 | | (3/4). |
| | | T47-D | | |
| | | [ER−] | | |
| 020626.01C | Neu+/+ x | Spontaneous | 10% RB IL | IL RB ablated spontaneous breast |
| | (1988A2K | Murine | 50 μL/tumor | tumors in mice. |
| | b x | | Qd × 1 | |
| | Neu+/+) | | | |

Hepatocellular Carcinoma Cell Lines

| | | | | |
|---|---|---|---|---|
| 020111.01B | BALB/c | Murine | 10% RB IL | IL RB completely ablated all |
| | Nude Mice | Hepa I-6 | 30-50 μL/tumor | tumors (8/8) with no damage to |
| | 8/group | | Qd × 1 | peripheral normal tissue (0/8) |
| | | | 95% EtOH IL | IL 95% EtOH did not induce |
| | | | 30-50 μL/tumor | complete |
| | | | Qd × 1 | tumor ablation (0/8) but induced |
| | | | | necrosis to peripheral normal |
| | | | | tissue (8/8) |
| 051016.02B | BALB/c | Murine | 10% RB | IL RB fully ablated 8/10 tumors 3 |
| | Nude Mice | Hepa 1-6 | 45 μL/tumor | days post injection. Partial |
| | 10/group | | Qd × 1 | responses were seen in 2 large |
| | | | | tumors. |

Other Cell Lines

| | | | | |
|---|---|---|---|---|
| 051017.01B | Nu/nu | H69Ar | 10% RB | IL RB fully ablated 5/5 tumors 24 |
| | Nude Mice | human | 45 μL/tumor | hours post injection and over 7 |
| | | resistant | Qd × 1 | days of observation. |
| | | SCLC | | |
| 051017.02B | Nu/nu | PC-3 | 10% RB | IL RB fully ablated 8/8 tumors 24 |
| | Nude Mice | human | 45 μL/tumor | hours post injection and over 29 |
| | | prostate | Qd × 1 | days of observation. |

ER+ represents estrogen-receptor positive cells, while ER− is estrogen-receptor negative cells.

5. In vivo Human Studies

A trial was conducted with 20 patients with at least two histologically or cytologically confirmed measurable melanoma lesions (stage III or IV metastatic melanoma). Each target lesion was 6 cm or less. All patients had normal haematopoietic, renal, hepatic and thyroid function. Patients receiving radiation therapy, chemotherapy, local treatment or other investigational agents within 4 weeks of study participation or an anti-tumor vaccine within 12 weeks were excluded, as were patients with acute concurrent illness.

### Clinical Protocol

Each patient received a single IL injection of a 10% RB solution in unbuffered saline. The pH of the solution was between about 6 to about 6.5. One to twenty target lesions were injected at a dose of 0.5 mL/cc lesion volume during a single treatment session. A 25 gauge needle was used for injection of large lesions; otherwise a 26 gauge or 30 gauge needle on a tuberculin syringe was used to allow price injection with minimal leakage from puncture sites when the needle was withdrawn. Injection was performed using a fanning technique to slowly and uniformly infiltrate the lesion with RB as the needle tip was inserted to the margin, then withdrawn along multiple tracks. In addition, one to three nontarget lesions were designated to assess potential bystander effect.

Patients were monitored for locoregional or systemic adverse effects and study lesions were followed for at last 12 weeks after RB injection to assess objective response.

The first 11 patients in this trial had 26 target lesions and 28 nontarget lesion in total, median age 83 years, range 75-86 years (data for the remaining 9 patients has not been completely analysed). They received RB injections into their target lesions (median V_{L} is 0.29 cm³, range 0.02-12.8 cm³) at single treatment sessions. Ten patients had locoregionally recurrent disease confined to a single lower extremity whereas the 11 the patient had locoregional disease of the head and neck. The mean dose of RB per patient was 1.6 mL (160 mg RB) with a range of 0.11-4.5 mL (11-45 mg RB,) whereas the mean dose injected into an individual lesion was 0.66 mL (range 0.01- 3.8 mL). Furthermore, one patient in the remaining 9 received 15 mL (i.e., 1500 mg RB). Patient characteristics, response to treatment and outcome are summarized in Table 7.

TABLE 7
Patient

| No/age | Target lesions | | | Nontarget lesions | | | Subsequent | Subsequent |
|---|---|---|---|---|---|---|---|---|
| and sex | Location | Size/mm | outcome | Location | Size | Outcome | history | status |
| 001, | Rt thigh | 13.0 | CR | right thigh | 15.0 | SD | Ex ITM + | Alive at 30 |
| 75, F | Rt lower | 16.0 | CR | right thigh | 10.0 | PD | DTIC; SD | months |
| | leg | | | | | | | |
| | Rt lower | 20.0 | CR | Rt lower leg | 17.0 | SD | | |
| | leg | | | | | | | |
| 002, | Lt lower | 10.1 | SD | Lt lower leg | 7.4 | CR | Regional | Alive at 34 |
| 77 F | leg | | | | | | node Ex(1+); | months |
| | Lt lower | 7.2 | PR | Lt lower leg | 8.1 | PR | NSR | |
| | leg | | | | | | | |
| | Lt lower | 12.7 | SD | Lt lower leg | 6.4 | PR | | |
| | leg | | | | | | | |
| 003, | Lt middle | 8.9 | PD | Lt upper leg | 7.7 | PD | ILI; died | Dead at 12 |
| 82 F | leg | | | | | | with MM | months |
| | Lt lower | 7.8 | PD | Lt thigh | 9.5 | PD | (SD) | |
| | leg | | | | | | | |
| 004, | Left thigh | 7.3 | PD | Lt thigh | 5.7 | PD | Ex ITM; ILI; | Dead at 9 |
| 84 F | Lt mid leg | 6.2 | PD | Lt thigh | 5.2 | PD | XRT; death | months |
| | Lt mid leg | 6.1 | PD | Lt middle leg | 5.1 | PD | from MM | |
| | | | | | | | (PD) | |
| 005 | Lt thigh | 9.5 | SD | Lt middle leg | 4.1 | SD | Ex ITM; | Alive at 31 |
| 83 F | Lt lower | 23.1 | PR | Lt lower leg | 5.7 | SD | NSR | months |
| | leg | | | | | | | |
| | Lt lower | 9.2 | CR | Lt foot | 6.8 | PD | | |
| | leg | | | | | | | |
| 006 | Rt mid leg | 6.0 | SD | Rt mid leg | 7.8 | PD | Ex ITM; ILI; | Alive at 27 |
| 78 F | Rt lower | | SD | Rt lower leg | 6.5 | ND | SD | months |
| | leg | | | | | | | |
| | Rt lower | | ND | Rt lower leg | 3.6 | ND | | |
| | leg | | | | | | | |
| 007 | Rt knee | 6.0 | SD | Rt knee | 9.6 | SD | Ex ITM; ILI' | Dead at 8 |
| 83 M | Rt Knee | 6.0 | PD | Rt lower leg | 6.7 | PD | death from | months |
| | Rt lower | 16.5 | SD | Rt lower leg | 8.8 | PD | MM (PD) | |
| | leg | | | | | | | |
| 008 | Rt knee | 26.8 | SD | Rt knee | 15.4 | SD | Ex ITM; PD | Alive at 28 |
| 76 M | | | | | | | | months |
| 009 | Lt knee | 29.0 | CR | Lt lower leg | 13.0 | SD | SD | Alive at 29 |
| 84 | Lt foot | | | | | | | months |
| 010 | Rt mid leg | 6.2 | CR | Rt thigh | 8.0 | PD | Death from | Dead at 9 |
| 84 M | Rt mid leg | 6.6 | PR | Rt mid leg | 6.6 | PR | MM (PD) | months |
| | | | | | | | | Rt midleg |
| 0109 | Left cheek | 17.2 | CR | Left ear | 36.0 | CR | NSR | Alive at 28 |
| 86M | | | | Lt upper neck | 27.0 | CR | | months |
| | | | | Lt lower neck | 9.0 | CR | | |

Abbreviations used in the table: DTIC, dacarbazine, Ex, excision, F, female, ILI, isolated limb infusion; ITM, Intransit metastasis; Lt, left; M, male; MM metastatic melanoma; ND, not done; NSR, no sign of recurrence; 1+, one positive node; PD, progressive disease; Rt, right; SD stable disease; XRT, radiation therapy.

### Clinical Response

Of the 26 target lesions treated with RB, 25 were evaluable at final follow up. Nine target lesions (36%) showed complete response (CR) based upon the Response Evaluation Criteria in Solid Tumors (RECIST) criteria (i.e., disappearance of lesion, histologically negative residual mass, or negative contrast-enhanced computed tomography scan at final follow up), three (12%) showed partial response (PR), seven (28%) showed stable disease and six (24%) showed progressive disease, for an objective response rate (ORR) of 48%.

A similar rate was noted for overall response analysed per patient [55% ORR based on 27% CR, 27% PR, 27% stable disease (SD) and 18% progressive disease (PD)].

Of 28 nontarget lesion assessed at screening, 26 were evaluable at final follow up. Non-target lesions exhibited an individual ORR of 27% (15% CR, 12% PR, 31% SD and 52% PD) that when analysed on a per patient basis also equalled 27% (9% CR, 18% PR, 45% SD and 27% PD). A strong positive correlation was noted between response of target and nontarget lesions, consistent with a possible bystander effect (correlation coefficient 0.803, P=0.003, N=11). Notably patients experiencing a positive objective response of target lesions exhibited a significantly higher rate of response in their nontarget lesions (44% ORR) than those with a negative lesion response (0%).

Lesions exhibiting CR or PR were characterized by an early onset of localized coagulative necrosis progressing to eschar within several days of injection. This was followed by gradual involution of the remaining tumor mass that appeared to continue beyond 12 weeks, with progressive improvement noted during longer-term follow up in several patients. This suggests that extended follow up could result in an increased ORR over that observed with this initial cohort. In contrast, lesions exhibiting disease progression demonstrated marked growth by 8 weeks, providing a clear basis for planning potential retreatment or alternative therapy for non-responsive lesions.

### Conclusions

In contrast to prior art IL treatments, a single treatment according to the method of the present invention, resulted in successful chemoablation in 48% of injected tumors (having a median volume of 0.29 cm) and approached 70% (62% CR+8% PR) in larger lesions receiving 0.2 mL or greater doses of RB according to the method of the invention. This robust response was associated with generally mild and transient adverse effects.

It may be appreciated that selective and rapid uptake of RB by tumor cells is important to the efficacy of this treatment. The present inventors have surprisingly and unexpectedly discovered that the combination of pH and electrolyte concentration improves uptake by tumor cells, without having any apparent effect on normal cells. Rapidity of uptake is important to maximize local toxicity against neoplastic tissue and minimize the level of drug able to be removed via the liver. A still further surprising result is that a solution prepared according to these critical parameters is also stable. Stability is also important for manufacture, transport, storage and administration.

Whilst WO 02/05812 discusses the potential use of RB for the treatment of neoplastic disease in general, there is no suggestion at all as to the criticality of electrolyte concentration and pH. Significantly, this document teaches the use of a phosphate buffer which provides a solution pH of 7.4. The Kp of RB at this pH in phosphate buffered saline was observed to be 11.5. This may be compared to 66.7 in the absence of phosphate buffer, in which case the pH would have been between about 6 to about 6.5.

Xanthene dyes, including RB are present in neutral, monoanion and dianion forms as shown below. The amount of each form present depends upon solvent and pH. The pH studies described in Example 2 show that at low pH, the water insoluble lactone form predominates. The lactone form, however, shows by far the highest partitioning coefficient. It will be appreciated therefore, that there are a number of competing factors influencing stability, selectivity and efficacy. The present inventors have surprisingly discovered that formulations within specific parameters have the desired degree of stability, selectivity and efficacy. Such a result could not have been predicted from the prior work which suggests functionalization to change chemical partitioning or biological activity.

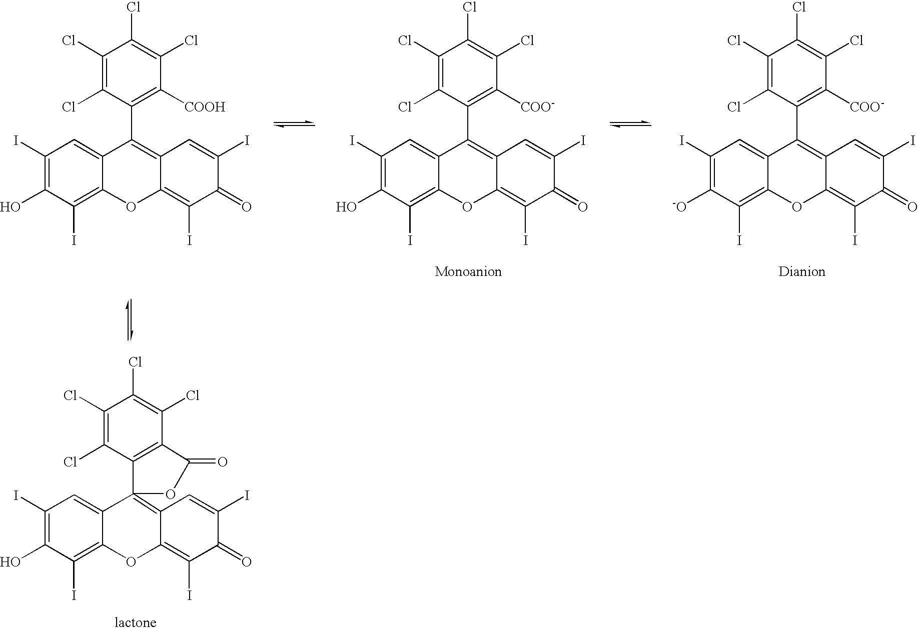

The method of the present invention finds particular application for the treatment of locoregionally recurrent disease. Locoregionally recurrent disease refers to recurrence of disease in the anatomical region from the primary site to the regional lymph nodes after apparently complete excision of the primary tumor.

In some cases, patients with locoregionally recurrent disease experience multiple and/or rapidly progressive lesions. In many cases, the number, size and recurrence mean that excision is not a viable option. This is of particular relevance for recurrent breast cancer and melanoma. Regional drug therapies such as isolated limb perfusion and isolated limb infusion are one option for treatment of locoregionally recurrent melanoma. A disadvantage is that these are surgical procedures with the associated risk of complications. In some cases, where the lesions are located on a proximal limb, trunk or head/neck regional therapy is not possible. The method of the present invention may find particular application for treatment of lesions of this type.

It will also be appreciated that the apparent bystander effect, evidenced by the response of non-treated lesions in the aforementioned clinical trial, indicates that intralesional treatment of cancer using the methods and medicaments described herein may have a systemic benefit that may lead to slowing, arrest or reversal of cancers located elsewhere in the body. Such systemic benefit may include increased period of progression free survival or increased overall survival resulting from localized treatment of one or more injected lesions. The present inventors speculate that such systemic benefit may accrue due to stimulation of the patient's immune system upon chemoablation of injected lesions, however the ultimate cause of this benefit may be found to arise via a different mechanism.

It will be appreciated that various changes and modifications may be made to the invention as described and claimed herein, without departing from the spirit and scope thereof

TABLE 4
Survival and outcome results for single treatment of A375 melanoma tumors. Animals
were followed until tumor reached 2.0 g or until conclusion of study.

| | | | | | | | | Outcome | | | |
|---|---|---|---|---|---|---|---|---|---|---|---|
| | | Regimen | | | | Survival | | | | #CR + | P |
| Group | N | Agent | | mg/kg | Route | MST ± SEM (n) | P (Log-Rank) | #CR | #PR/SD | #PR/SD (%) | (Fishers) |
| A | 18 | Growth Control | | n/a | n/a | 26.7 ± 2.6 (17) | n/a | 0 | 1 | 1 (6) | n/a |
| B | 6 | Saline | | n/a | IL | 27.6 ± 2.7 (6) | 0.742 | 0 | 0 | 0 (0) | 1.000 |
| C | 5 | 0.1% | RB | 5 | IL | 27.0 ± 3.0 (5) | 0.765 | 0 | 0 | 0 (0) | 1.000 |
| D | 12 | 1% | RB | 50 | IL | 33.1 ± 4.5 (10) | 0.226 | 1 | 1 | 2 (17) | 0.548 |
| E | 17 | 3% | RB | 150 | IL | 50.0 ± 7.0 (11) | 0.004 | 3 | 3 | 6 (35) | 0.041 |
| F | 6 | 6% | RB | 300 | IL | 35.4 ± 6.6 (5) | 0.155 | 1 | 0 | 1 (17) | 0.446 |
| G | 5 | 10% | RB | 500 | IL | 52.2 ± 14.9 (3) | 0.025 | 1 | 1 | 2 (40) | 0.107 |

MST: Mean survival time (days) to 2.0 g tumor
P (Log-Rank): P-value for Log-Rank Test of respective treatment group Survival Curve vs. Growth Control Survival Curve
P (Fishers): P-value for Fishers Exact Test of positive outcome (#CR + #PR/SD) vs. negative outcome for treatment group vs. Growth Control

1. A method for the treatment of metastatic melanoma, the method comprising intralesional administration of a hydrophilic vehicle comprising 4,5,6,7-Tetrachloro-2′,4′,5′,7′-tetraiodofluorescein, or a physiologically acceptable salt thereof, at a concentration of about 1 w/v % up to about 20 w/v % and an electrolyte comprising at least one cation selected from the group consisting of sodium potassium, calcium and magnesium and at least one anion selected from the group consisting of chloride, phosphates and nitrates, wherein the electrolyte is at a concentration of between about 0.1 w/v % and about 2 w/v % and the pH of the solution is between about 4 to about 10.

2. The method of claim 1, wherein the hydrophilic vehicle is water.

3. The method of claim 1, wherein the salt is selected from the group consisting of sodium, potassium, lithium, calcium, magnesium, barium ammonium and protamine zinc salts.

4. The method of claim 1, wherein the salt is the disodium salt.

5. The method of claim 1, wherein the electrolyte is sodium chloride.

6. The method of claim 1, wherein the concentration of the electrolyte is between about 0.5% to about 1.5%.

7. The method of claim 6, wherein the concentration of the electrolyte is about 0.9%.

8. The method of claim 1, wherein the concentration of 4,5,6,7-Tetrachloro-2′,4′,5′,7′-tetraiodofluorescein is between about 3% to about 10%.

9. The method of claim 1, wherein the concentration of 4,5,6,7-Tetrachloro-2′,4′,5′,7′-tetraiodofluorescein is about 10%.

10. The method of claim 1, wherein the hydrophilic vehicle does not contain a buffer.

11. The method of claim 1, wherein the pH is between about 5 to about 7.

12. The method of claim 1, wherein the pH is between about 6 to about 6.8.

13. The method of claim 1, wherein the lesion is associated with stage III or stage IV disease.

14. The method of claim 11, wherein the lesion is a selected from the group consisting of liver cancer or breast cancer.

15. The method of claim 1, wherein the lesion is a locoregionally recurrent lesion.

16. The method of claim 1, wherein between 0.1 mL/cc lesion volume to about 2 mL/cc lesion volume is administered.

17. The method of claim 1, wherein between about 0.25 mL/cc lesion volume to about 0.75 mL/cc lesion is administered.

18. A method for the treatment of a neoplastic lesion, the method comprising intralesional administration of a hydrophilic vehicle comprising 4,5,6,7-Tetrachloro-2′,4′,5′,7′-tetraiodofluorescein, or a physiologically acceptable salt thereof, at a concentration of about 0.1 w/v % up to about 20 w/v % and an electrolyte comprising at least one cation selected from the group consisting of sodium, potassium, calcium and magnesium and at least one anion selected from the group consisting of chlorine, a phosphate and a nitrate, wherein the electrolyte is at a concentration of between about 0.1 w/v % and about 2 w/v % and the pH of the solution is between about 4 to about 10.

19. The method of claim 18, wherein the lesion is a selected from the group consisting of liver cancer or breast cancer.

20. The method of claim 18, wherein the lesion is a locoregionally recurrent lesion.

21. The method of claim 18, wherein between 0.1 mL/cc lesion volume to about 2 mL/cc lesion volume is administered.

22. The method of claim 18, wherein between about 0.25 mL/cc lesion volume to about 0.75 mL/cc lesion is administered.

23. Use of a 4,5,6,7-Tetrachloro-2′,4′,5′,7′-tetraiodofluorescein, or a physiologically acceptable salt thereof, in the preparation of a medicament for the treatment of a neoplastic lesion, wherein the medicament comprises a hydrophilic vehicle comprising 4,5,6,7-Tetrachloro-2′,4′,5′,7′-tetraiodofluorescein at a concentration of about 0.1 w/v % up to about 20 w/v %, an electrolyte comprising at least one cation selected from the group consisting of sodium, potassium, calcium and magnesium and at least one anion selected from the group consisting of chlorine, a phosphate and a nitrate, a concentration of between about 0.1 w/v % and about 2 w/v % and the pH of the solution is between about 4 to about 10.
